(19) United States
(12) Patent Application Publication
(10) Pub. No.: US 2024/0325693 A1
(43) Pub. Date: Oct. 3, 2024

(54) EXTENDABLE TUBING EXTENSION SETS

(71) Applicant: CareFusion 303, Inc., San Diego, CA (US)

(72) Inventor: Jason Andrew WINE, Brea, CA (US)

(21) Appl. No.: 18/585,651

(22) Filed: Feb. 23, 2024

Related U.S. Application Data
(60) Provisional application No. 63/493,262, filed on Mar. 30, 2023.

(51) Int. Cl. A61M 25/02; A61M 5/14

(52) U.S. Cl. CPC A61M 25/02; A61M 5/1415

(57) ABSTRACT

Extendable tubing extension sets are disclosed having a tubing cradle with first and second ends that can be positioned between un-extended and extended configurations, where in the un-extended configuration, the first and second ends of the tubing cradle are a first distance apart, where the first distance is less than a length from the first end of the tubing cradle to the second end of the tubing cradle, and in the extended configuration, the first and second ends are positioned a second distance apart, where the second distance is greater than the first distance, and where the tubing cradle is configured to receive a length of tubing therein such that the extendable tubing extension can resist separation or disconnection of a fluid pathway through the tubing by moving from the un-extended to the extended configuration.

### CROSS-REFERENCE TO RELATED APPLICATIONS

This application claims priority under 35 U.S.C. § 119 to Provisional Application Ser. No. 63/493,262, filed Mar. 30, 2023, entitled “EXTENDABLE TUBING EXTENSION SETS”, the disclosure of which is incorporated herein by reference in its entirety for all purposes.

### BACKGROUND

The present disclosure relates generally to medical fluid connectors and, more particularly, to extendable tubing extension sets that are movable between an un-extended and extended configuration.

Medical connections are widely used in fluid delivery systems such as those used in connection with intravenous (IV) fluid lines, blood access, hemodialysis, peritoneal dialysis, enteral feeding, drug vial access, and other procedures.

In some instances, the medical connection can become dislodged or disconnected in an unintended manner. For example, medical tubing of an IV set that is coupled to a catheter can become dislodged when an unintended or unexpected forces is exerted upon a portion of the IV set such as the catheter or the tubing, which may exceed the design limitations of the catheter securement method or another portion of the IV set. An unintended or unexpected force can be applied to the tubing and/or catheter when the patient moves or rolls over within a bed, or when the tubing or another portion of an intravenous set become caught on a portion of the bed, such as the railing, or when a patient is panicking, disoriented, or fidgeting to such an extent that the medical tubing is unintentionally or intentionally pulled away from the patient or away from the medical equipment coupled to the tubing.

### SUMMARY

In accordance with at least some embodiments disclosed herein is the realization that unintended dislodgement or disconnection of a medical connection, such as a medical tubing, can result in injury to a patient or a caregiver, such as by depriving the patient of a medicament, increasing the potential for infection to the patient, and exposing the caregiver to harmful medicaments.

Accordingly, aspects of the present disclosure provide an extendable tubing extension set comprising a tubing cradle comprising a first end, a second end, and a tubing passage extending through the first and second ends of the tubing cradle, wherein the tubing cradle comprises a tubing cradle length from the first end to the second end of the tubing cradle, an un-extended configuration wherein the first end of the tubing cradle is positioned a first distance from the second end of the tubing cradle, and the first distance is less than the tubing cradle length, and an extended configuration wherein any of the first end or the second end of the tubing cradle are moved in a direction away from the other of the first end and the second end, such that the first end of the tubing cradle is positioned a second distance from the second end of the tubing cradle, and the second distance is greater than the first distance.

The present disclosure can also provide an extendable tubing extension set comprising an extendable tubing extension set comprising a tubing cradle comprising a first end, a second end, and a tubing passage extending through the first and second ends of the tubing cradle, wherein the tubing cradle comprises a tubing cradle length from the first end to the second end of the tubing cradle, a tubing comprising a proximal end portion, a distal end portion, and a middle portion, wherein the middle portion of the tubing is positioned within the tubing passage with the proximal end portion of the tubing extending beyond the first end of the tubing cradle and the distal end portion of the tubing extending beyond the second end of the tubing cradle, an un-extended configuration wherein the first end of the tubing cradle is positioned a first distance from the second end of the tubing cradle, and the first distance is less than the tubing cradle length, and an extended configuration wherein any of the first end or the second end are moved in a direction away from the other of the first end and the second end, such that the first end of the tubing cradle is positioned a second distance from the second end of the tubing cradle, and the second distance is greater than the first distance.

Accordingly, the present application addresses several operational challenges encountered in prior fluid connections and provides numerous improvements that enable the user to more easily provide a fluid pathway, and to increase safety and efficacy in maintaining a fluid pathway and fluid delivery. In some aspects, the present disclosure provides an extendable tubing extension set that can resist separation or damage to a fluid pathway by providing a tubing cradle that can move from un-extended configuration and an extended configuration.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1A illustrates an extendable tubing extension set in an un-extended configuration in use with an IV set coupled to a patient, in accordance with aspects of the present disclosure.

FIG. 1B illustrates an extendable tubing extension set in an extended configuration in use with an IV set coupled to a patient, in accordance with aspects of the present disclosure.

FIG. 2 illustrates a top perspective view of an extendable tubing extension set in an un-extended configuration, in accordance with aspects of the present disclosure.

FIG. 3 illustrates a perspective view of the extendable tubing extension set of FIG. 2 in an extended configuration, in accordance with aspects of the present disclosure.

FIG. 4 illustrates a bottom view of an extendable tubing extension set, in accordance with aspects of the present disclosure.

FIG. 5 illustrates a top view of an extendable tubing extension set, in accordance with aspects of the present disclosure.

FIG. 6 illustrates a top view of an extendable tubing extension set, in accordance with aspects of the present disclosure.

FIG. 7 illustrates a first side view of the extendable tubing extension set of FIG. 6.

FIG. 8 illustrates a second side view of the extendable tubing extension set of FIG. 6.

FIG. 9 illustrates a perspective view of an extendable tubing extension set, in accordance with aspects of the present disclosure.

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

In accordance with some embodiments, the present application discloses various features and advantages of an extendable tubing extension set. The extendable tubing extension set can provide for efficient and safe maintenance of a fluid pathway, such as a fluid pathway used for transferring medical fluids toward or away from a patient. The extendable tubing extension set can maintain the structural integrity and sterility of a fluid pathway by resisting unintended disconnection when a pulling or tension force is applied to the extendable tubing extension set or another device that forms the fluid pathway, such as when a patient moves or when the medical tubing is pulled away from the patient.

The extendable tubing extension set can also prevent injury to a patient or a caregiver by preventing unintended disconnection of the fluid pathway from the patient, which could deprive the patient of a medicament and could expose the patient or another person to the medicament. The extendable tubing extension set can also reduce labor and cost associated with fluid transfer by avoiding unintended disconnection of the fluid pathway.

The extendable tubing extension set is configured to be used, at least initially, with a patient in an un-extended configuration and is configured to move to an extended configuration if a force that exceeds a threshold is exerted on the extendable tubing extension set or on a tubing coupled to the extendable tubing extension set.

If a force that exceeds a threshold is exerted on the extendable tubing extension set or on a tubing coupled to the extendable tubing extension set, the tubing cradle can move from a gathered or coiled position in the un-extended configuration, to an extended or uncoiled position in the extended configuration. Thus, the extendable tubing extension set can function as a buffer by providing an additional length of movable fluid pathway in the event that the tubing is pulled to an extent or force that would have otherwise caused dislodgment or separation of the tubing without the buffer.

In the un-extended configuration, the tubing cradle forms a pattern that results in the first and second ends of the tubing cradle being positioned a distance apart that is less that the length from the first end to the second end of the tubing cradle. When the tubing cradle is un-gathered or uncoiled in the extended configuration, the distance between the first and second ends of the tubing cradle is increased relative to the distance between the first and second ends in the un-extended configuration. Because the tubing cradle is configured to receive a length of tubing therein, the length of tubing remains the same between the un-extended and extended configurations, but the distance between proximal and distal ends of the tubing increases when the tubing cradle is moved from the un-extended configuration toward the extended configuration.

To resist movement from the un-extended configuration to the extended configuration, the tubing cradle can be formed or made in the shape or position of the un-extended configuration. In the un-extended configuration, the strength of the material forming the tubing cradle can resist movement to the extended configuration. The extendable tubing extension set can also be configured with connections between portions of the tubing cradle to resist movement to the extended configuration. The connections between portions of the tubing cradle can be formed by any of an adhesive, weld, or structure between the portions of the tubing cradle. The connections between portions of the tubing cradle can be configured to separate and/or break to permit the tubing cradle to move from the un-extended configuration to the extended configuration. In some embodiments of the present disclosure, the extendable tubing extension set can have a base and portions of the tubing cradle can be coupled to the base resist movement to the extended configuration. In some aspects of the present disclosure, the base is configured to be coupled to a patient.

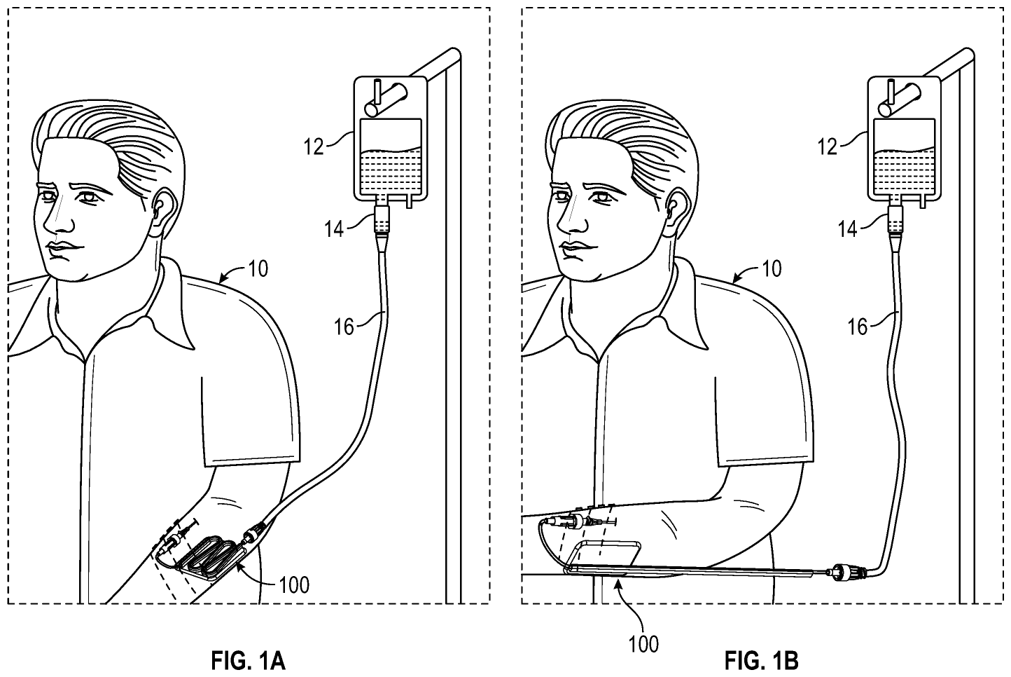

An example of the extendable tubing extension set 100 is shown with a patient, in the un-extended configuration, in FIG. 1A, and the in the extended configuration in FIG. 1B. The extendable tubing extension set is coupled with tubing of an IV set, which is being used to direct a fluid to a patient 10. The IV set can include a medicament bag 12, a drip chamber 14, tubing 16, and an IV catheter 18. In the un-extended and extended configurations, the extendable tubing extension set 100 forms a fluid pathway having a length that remains the same in the un-extended and extended configurations.

In the un-extended configuration shown in FIG. 1A, the extendable tubing extension set 100 is coupled to an arm of the patient and the tubing cradle is gathered to form a zig-zag pattern having alternating direction such that the first and second ends of the tubing cradle are spaced apart by a first distance that is less than the length from the first end to the second end of the tubing cradle. In FIG. 1B, the patient's arm has been moved in a direction away from the IV set, which would have otherwise caused disconnection of the tubing from the IV set; however, the patient's movement exerted a force on the tubing and extendable tubing extension set 100 that caused the extendable tubing extension set to move from the un-extended configuration to the extended configuration. In the extended configuration, the first and second ends of the tubing cradle are spaced apart by a second distance that is greater that the first distance, thereby maintaining the fluid pathway between the patient 10 and the medicament bag 12.

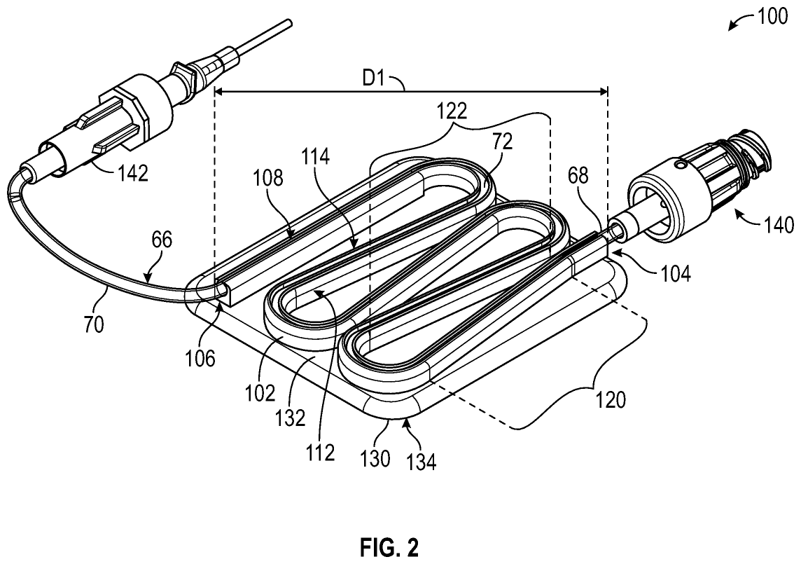

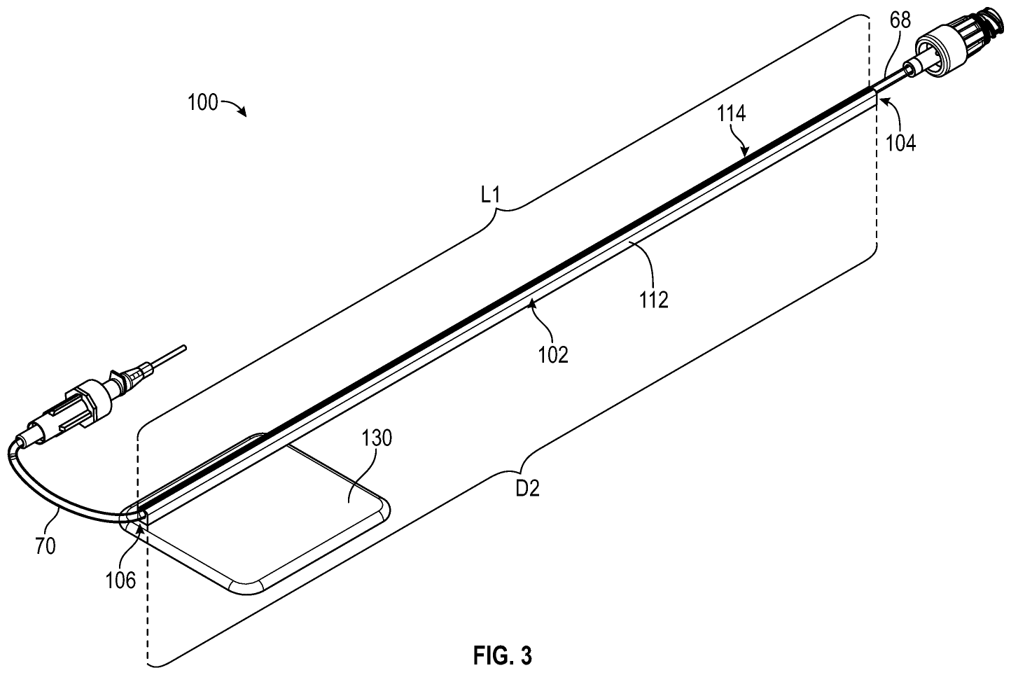

An embodiment of the extendable tubing extension set 100 in the un-extended configuration is shown in FIG. 2, and in the extended configuration is shown in FIG. 3. The extendable tubing extension set includes a tubing cradle 102 having a first end 104, a second end 106, and a tubing passage 108 extending through the first and second ends 104, 106 of the tubing cradle.

A length of tubing 66 is coupled to the tubing cradle 102 and extends along the tubing passage 108 so that the tubing 66 can move together with the tubing cradle 102 between the un-extended and extended configurations. The tubing 66 includes a proximal end portion 68, a distal end portion 70, and a middle portion 72 extending between the proximal and distal end portions. When coupled to the tubing cradle 102, the middle portion 72 of the tubing extends through the tubing passage 108, the proximal end portion 68 of the tubing extending beyond the first end 104 of the tubing cradle, and the distal end portion 70 of the tubing extending beyond the second end 106 of the tubing cradle.

The tubing cradle 102, and the tubing therein, are oriented so that the first end of the tubing cradle is positioned a first distance D1 from the second end of the tubing cradle, and the first distance is less than a tubing cradle length L1, where the tubing cradle length L1 is the length from the first end 104 to the second end 106 of the tubing cradle. To provide the un-extended configuration, where the first distance D1 between the first and second ends 104, 106 of the tubing cradle is less than the tubing cradle length L1, the tubing cradle 102 extends in more than one direction between the first and second ends 104, 106 of the tubing cradle.

In the embodiment shown in FIG. 1, the tubing cradle 102 extends in a back-and-forth or zig-zag pattern between the first and second ends 104, 106 of the tubing cradle. In the back-and-forth or zig-zag pattern, a first portion 120 of the tubing cradle that is between the first end 104 and the second end 106 extends in a first direction, and a second portion 122 of the tubing cradle that is between the first portion 120 and the second end 106 of the tubing cradle can extend in a second direction that is different than the first direction.

Although the tubing cradle 102 is shown having a back-and-forth or zig-zag pattern, it should be understood that the present disclosure contemplates embodiments in which the tubing cradle 102 can form any pattern between the first and second ends 104, 106 of the tubing cradle such that the first distance D1 between the first and second ends 104, 106 of the tubing cradle is less than the tubing cradle length L1.

In some embodiments of the present disclosure, the tubing cradle 102 can form a coil that extends in any of a clockwise or counterclockwise direction between the first and second ends 104, 106 of the tubing cradle when in the un-extended configuration. In some embodiments, the tubing cradle 102 forms a spiral having a regular shape, such as a circle or square, or irregular shape.

To provide the un-extended configuration, the tubing cradle 102 can be formed monolithically formed or manufactured with the back-and-forth or zig-zag pattern. In some aspects of the present disclosure, the formation or manufacturing of the tubing cradle 102 provides the material strength to resist movement to the extended configuration.

The tubing cradle 102 is configured to retain the tubing in a tubing passage 108. The tubing passage 108 can be formed by a first longitudinal wall 112 and a second longitudinal wall 114, where the first and second longitudinal walls extending coaxially between the first and second ends 104, 106 of the tubing cradle. To retain the tubing 66, the first and second longitudinal walls 112, 114 are spaced to form the tubing passage 108 between an inner surface of the first longitudinal wall 112 and an inner surface of the second longitudinal wall 114.

In some embodiments of the present disclosure, any of the first and second longitudinal walls 112, 114 form discontinuous portions along the tubing cradle length L1. The discontinuous portions of the tubing cradle can provide flexibility for the tubing cradle 102 to be bent or oriented in the gathered or coiled position in the un-extended configuration.

The discontinuous portions of the tubing cradle can be formed by channels or passages that extend through any of the first longitudinal wall 112 and/or the second longitudinal wall 114. The extendable tubing extension set 100 can be configured so that discontinuous portions of the first longitudinal wall 112 are longitudinally aligned with discontinuous portions of the second longitudinal wall 114. The extendable tubing extension set 100 can also be configured, in some embodiments, so that the discontinuous portions of the first longitudinal wall 112 are longitudinally misaligned with discontinuous portions of the second longitudinal wall 114.

To couple the extendable tubing extension set 100 to a patient, tape can be placed over a top surface of tubing cradle and adhered to the patient to resist movement of the extendable tubing extension set 100, relative to the patient, in the un-extended configuration. The tubing cradle 102, in some embodiments, can have an adhesive along a bottom surface configured to engage against the patient.

In some embodiments, the extendable tubing extension set 100 includes a base 130 configured to retain the tubing cradle 102 and to be engaged against the patient. The base has a top surface 132 and a bottom surface 134, wherein the bottom surface 134 is configured to engage against a patient, and the top surface 132 is configured to engage against the tubing cradle.

To retain the tubing cradle 102, the base 130 and tubing cradle 102 can be coupled together along a portion of the tubing cradle length L1. For example, a portion of the tubing cradle length L1, extending from the second end 106 of the tubing cradle toward the first end 104 of the tubing cradle can be coupled to or formed with the base 130.

In the un-extended configuration, the first and second ends 1024, 106 of the tubing cradle are positioned over the top surface 132 of the base. When the extendable tubing extension set 100 is moved toward the extended configuration, the first end 104 of the tubing cradle is moved relative to the base 130. In the extended configuration, the first end 104 of the tubing cradle extends beyond the perimeter of the base 130. In some embodiments, any of the first and/or second ends of the cradle can be configured move relative to the base 130 in the extended configuration.

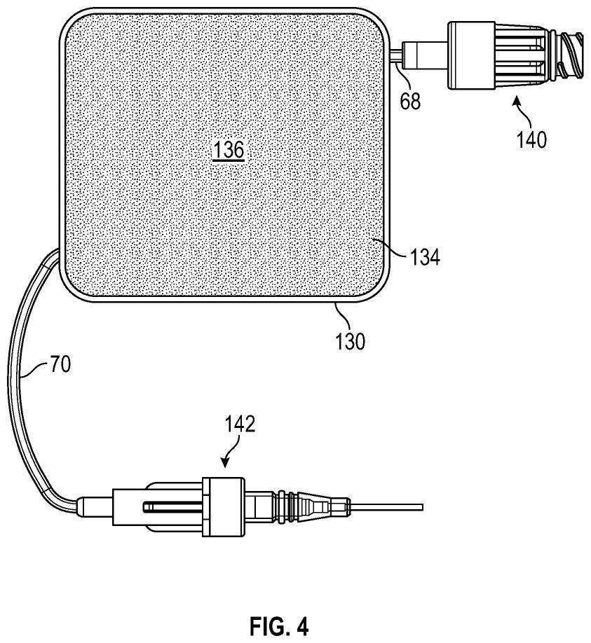

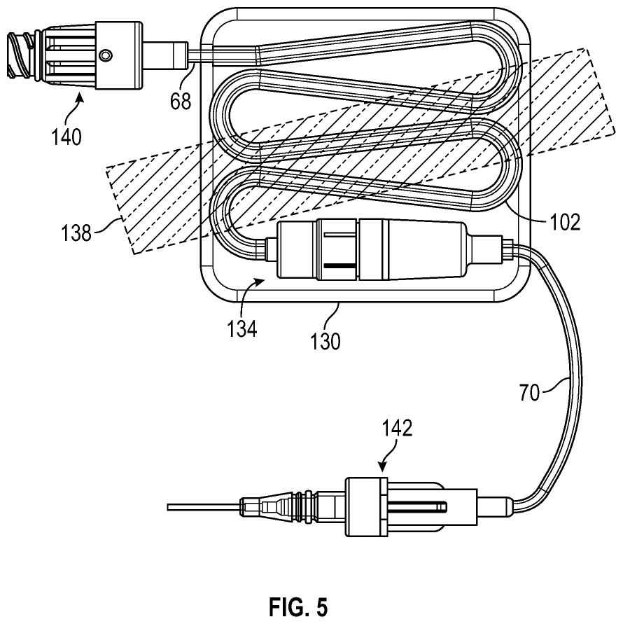

The base 130 can also provide a surface for tape or an adhesive to be applied for coupling the extendable tubing extension set 100 to a patient. Referring to FIG. 4, which shows the bottom surface 134 of the base, the bottom surface 134 of the comprises an adhesive that is configured to bond to a patient in some embodiments of the present disclosure. Referring to FIG. 5, the extendable tubing extension set 100 is shown with tape 138 extending over the tubing cradle 102 couple the extendable tubing extension set 100 with a patient.

The extendable tubing extension set 100 can, in some embodiments, include a first fluid connector 140 coupled to a proximal end of the tubing 66, and a second fluid connector 142 coupled to a distal end of the tubing. Because the proximal end portion 68 of the tubing extends away from the first end 104 of the tubing cradle, and the distal end portion 70 of the tubing extends away from the second end 106 of the tubing cradle, the proximal and distal end portions 68, 70 of the tubing are movable relative to the tubing cradle. Because the first and second fluid connectors 140, 142 are coupled to the first and second end portions 68, 70 of the tubing, the tubing extension set 100 can permit the first and second end portions 68, 70 to move to resist separation of the extendable tubing extension set 100 or disconnection of the fluid passageway.

In use, if the tubing 66 or a portion of the extendable tubing extension set 100 is pulled beyond a first threshold force or distance, either of the first or second end 104, 106 of the tubing cradle can move relative to the other of the first or second end 104, 106 of the tubing cradle such that the extendable tubing extension set 100 moves from the un-extended to the extended configuration.

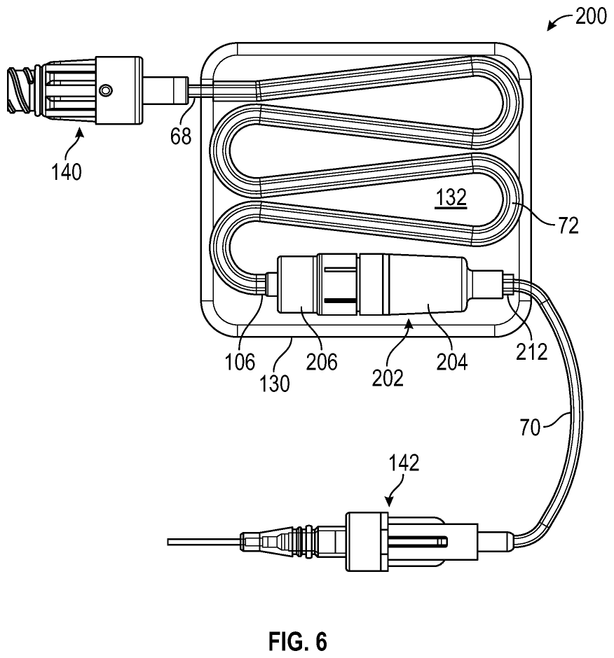

In some embodiments of the present disclosure, such as the embodiment of the extendable tubing extension set 200 shown in FIGS. 6 to 8, the extendable tubing extension set 200 can include a fluid disconnection device 202 fluidly coupled to the tubing 66. The fluid disconnection device 202 is configured to separate the fluid flow path between the first end portion 68 and the second end portions 70 of the tubing when a force exerted on the tubing 66 or portion of the extendable tubing extension set 200 exceeds a second threshold, where the second threshold is greater than the first threshold. Thus, the fluid disconnection device 202 can separate the fluid flow path after the fluid disconnection device 202 is moved to extended configuration.

To permit separation of the fluid flow path, the fluid disconnection device 202 includes a first portion 204 and a second portion 206 that are removably coupled together. When the first and second portions 204, 206 of the fluid disconnection device are coupled together, a fluid flow path through the fluid disconnection device 202 is open so that fluid can flow between the first end portion 68 and the second end portions 70 of the tubing. When the first and second portions 204, 206 of the fluid disconnection device are separated from each other, the portion of the fluid flow path within each of the first and second portions 204, 206 of the fluid disconnection device can become obstructed.

The fluid disconnection device 202 is coupled to the tubing 66, between the middle portion 72 of the tubing and the distal end portion of the tubing 70, and between the second end 106 of the tubing cradle and a distal end of the tubing. However, the present disclosure also contemplates that the fluid disconnection device 202 can be coupled to any portion of the tubing 66, such as the proximal end portion 68 or the distal end portion 70 of the tubing.

In use, when a force is exerted on the tubing 66 or portion of the extendable tubing extension set 200 that exceeds the second threshold, the fluid disconnection device 202 permits the second end portion 70 of the tubing and the second fluid connector 142 to remain coupled to the patient, thereby resisting injury to the patient or decoupling of the intravenous fluid pathway from the patient. In some aspects of the present disclosure, when the second threshold is exceeded, the first end portion 68 and the middle portion 72 of the tubing, and the second portion 206 of the fluid disconnection device, can separate from the first portion 204 of the fluid disconnection device.

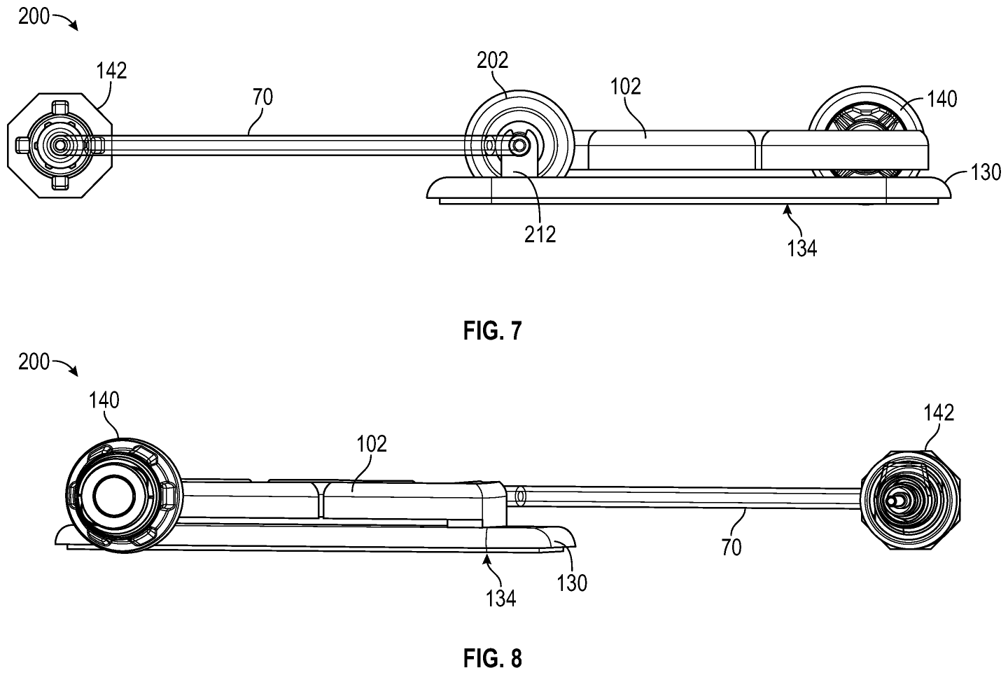

In some embodiments of the present disclosure, the base includes an arm 212 that extends from the base 130 to couple with a portion of the tubing 66 to resist movement of the portion of tubing relative to the base. As shown in FIGS. 6 and 7, for example, the arm 212 can removably couple to the distal end portion 70 of the tubing, between the fluid disconnection device 202 and the distal end of the second fluid connector 142. When the second threshold is exceeded, the tubing can decouple from the arm 212 to permit the base 130 to separate from the distal end portion 70 if the tubing.

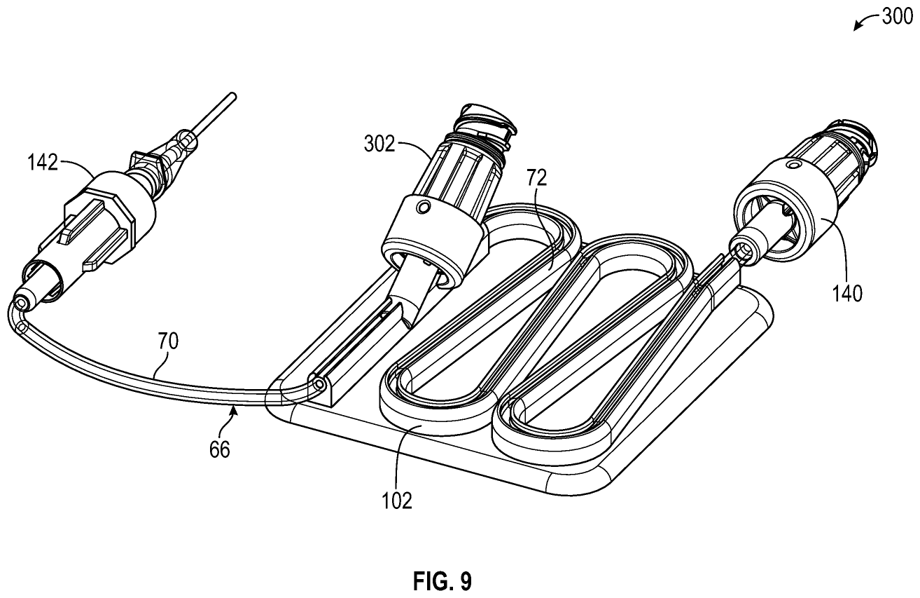

FIG. 9 illustrates another embodiment of an extendable tubing extension set 300 comprising an auxiliary port 302 fluidly coupled to the tubing 66 and configured to permit a fluid to injected into the fluid pathway without disconnecting another portion of the fluid pathway, such as the first or second fluid connectors 140, 142. The auxiliary port 302 also permits injection into the fluid pathway without affecting the priming volume of the extendable tubing extension set 300.

In some embodiments of the present disclosure, the auxiliary port 302 is formed by a y-connection along a portion of the tubing 66. In some embodiments, the y-connection is positioned between the middle portion 72 and the distal end portion 70 of the tubing. In some aspects, the auxiliary port includes a needleless connector.

The features of the present disclosure provide embodiments of extendable tubing extension sets that can resist separation or disconnection of the fluid pathway when the extendable tubing extension set is in un-extended and extended configurations. In un-extended configurations, features such as the proximal end portion and distal end portion of the tubing extending away from the tubing cradle can resist separation or disconnection of the fluid pathway when a force is below a first threshold. Further, features including any of the structure of the tubing cradle and/or coupling between the tubing cradle and the base can resist separation or disconnection of the fluid pathway when a force is below a first threshold. When a force exceeds a second threshold, features such as the fluid disconnection device can permit intended disconnection of the fluid pathway and separation of a portion of the extendable tubing extension set to prevent injury to the patient and prevent leakage of the fluid from the tubing.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1. An extendable tubing extension set comprising: a tubing cradle comprising a first end, a second end, and a tubing passage extending through the first and second ends of the tubing cradle, wherein the tubing cradle comprises a tubing cradle length from the first end to the second end of the tubing cradle; an un-extended configuration wherein the first end of the tubing cradle is positioned a first distance from the second end of the tubing cradle, and the first distance is less than the tubing cradle length; and an extended configuration wherein any of the first end or the second end of the tubing cradle are moved in a direction away from the other of the first end and the second end, such that the first end of the tubing cradle is positioned a second distance from the second end of the tubing cradle, and the second distance is greater than the first distance.

Clause 2. The extendable tubing extension set of Clause 1, wherein the tubing cradle comprises a first longitudinal wall and a second longitudinal wall, the first and second longitudinal walls extending coaxially between the first and second ends of the tubing cradle and are spaced apart such that the tubing passage is formed between an inner surface of the first longitudinal wall and an inner surface of the second longitudinal wall.

Clause 3. The extendable tubing extension set of Clause 2, wherein any of the first and second longitudinal walls form discontinuous portions along the tubing cradle length.

Clause 4. The extendable tubing extension set of Clause 3, wherein the discontinuous portions of the first longitudinal wall are longitudinally aligned with the discontinuous portions of the second longitudinal wall.

Clause 5. The extendable tubing extension set of Clause 3, wherein the discontinuous portions of the first longitudinal wall are misaligned with the discontinuous portions of the second longitudinal wall.

Clause 6. The extendable tubing extension set of Clause 2, wherein the tubing cradle is monolithically formed in the un-extended configuration.

Clause 7. The extendable tubing extension set of Clause 2, wherein, in the un-extended configuration, a first portion of the tubing cradle length extends in a first direction, and a second portion of the tubing cradle length extends in a second direction, where the second direction is different than the first direction.

Clause 8. The extendable tubing extension set of Clause 1, comprising a base having a top surface and a bottom surface, wherein the bottom surface is configured to engage against a patient, and the top surface is configured to engage against the tubing cradle.

Clause 9. The extendable tubing extension set of Clause 8, wherein, in the un-extended configuration, the first and second ends of the tubing cradle are positioned over the top surface of the base.

Clause 10. The extendable tubing extension set of Clause 8, wherein the tubing cradle is movable, relative to the base, along a first portion of the tubing cradle length.

Clause 11. The extendable tubing extension set of Clause 10, wherein the tubing cradle is coupled to the base along a second portion of the tubing cradle length.

Clause 12. The extendable tubing extension set of Clause 8, comprising an adhesive along at least a portion of the bottom surface of the base.

Clause 13. An extendable tubing extension set comprising: a tubing cradle comprising a first end, a second end, and a tubing passage extending through the first and second ends of the tubing cradle, wherein the tubing cradle comprises a tubing cradle length from the first end to the second end of the tubing cradle; a tubing comprising a proximal end portion, a distal end portion, and a middle portion, wherein the middle portion of the tubing is positioned within the tubing passage with the proximal end portion of the tubing extending beyond the first end of the tubing cradle and the distal end portion of the tubing extending beyond the second end of the tubing cradle; an un-extended configuration wherein the first end of the tubing cradle is positioned a first distance from the second end of the tubing cradle, and the first distance is less than the tubing cradle length; and an extended configuration wherein any of the first end or the second end are moved in a direction away from the other of the first end and the second end, such that the first end of the tubing cradle is positioned a second distance from the second end of the tubing cradle, and the second distance is greater than the first distance.

Clause 14. The extendable tubing extension set of Clause 13, comprising a first fluid connector coupled to a proximal end of the tubing, and a second fluid connector coupled to a distal end of the tubing.

Clause 15. The extendable tubing extension set of Clause 13, comprising a fluid disconnection device coupled to the tubing.

Clause 16. The extendable tubing extension set of Clause 15, wherein the fluid disconnection device is coupled between the middle portion of the tubing and the distal end portion of the tubing, between the second end of the tubing cradle and a distal end of the tubing.

Clause 17. The extendable tubing extension set of Clause 15, wherein the fluid disconnection device is positioned between the second end of the tubing cradle and a distal end of the tubing.

Clause 18. The extendable tubing extension set of Clause 13, comprising a base having a top surface and a bottom surface, wherein the bottom surface is configured to engage against a patient, and the top surface is configured to engage against the tubing cradle.

Clause 19. The extendable tubing extension set of Clause 18, wherein the base comprises an arm configured to couple with the tubing.

Clause 20. The extendable tubing extension set of Clause 18, comprising an auxiliary port coupled to the tubing.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. An extendable tubing extension set comprising:
a tubing cradle comprising a first end, a second end, and a tubing passage extending through the first and second ends of the tubing cradle, wherein the tubing cradle comprises a tubing cradle length from the first end to the second end of the tubing cradle;
an un-extended configuration wherein the first end of the tubing cradle is positioned a first distance from the second end of the tubing cradle, and the first distance is less than the tubing cradle length; and
an extended configuration wherein any of the first end or the second end of the tubing cradle are moved in a direction away from the other of the first end and the second end, such that the first end of the tubing cradle is positioned a second distance from the second end of the tubing cradle, and the second distance is greater than the first distance.

2. The extendable tubing extension set of claim 1, wherein the tubing cradle comprises a first longitudinal wall and a second longitudinal wall, the first and second longitudinal walls extending coaxially between the first and second ends of the tubing cradle and are spaced apart such that the tubing passage is formed between an inner surface of the first longitudinal wall and an inner surface of the second longitudinal wall.

3. The extendable tubing extension set of claim 2, wherein any of the first and second longitudinal walls form discontinuous portions along the tubing cradle length.

4. The extendable tubing extension set of claim 3, wherein the discontinuous portions of the first longitudinal wall are longitudinally aligned with the discontinuous portions of the second longitudinal wall.

5. The extendable tubing extension set of claim 3, wherein the discontinuous portions of the first longitudinal wall are misaligned with the discontinuous portions of the second longitudinal wall.

6. The extendable tubing extension set of claim 2, wherein the tubing cradle is monolithically formed in the un-extended configuration.

7. The extendable tubing extension set of claim 2, wherein, in the un-extended configuration, a first portion of the tubing cradle length extends in a first direction, and a second portion of the tubing cradle length extends in a second direction, where the second direction is different than the first direction.

8. The extendable tubing extension set of claim 1, comprising a base having a top surface and a bottom surface, wherein the bottom surface is configured to engage against a patient, and the top surface is configured to engage against the tubing cradle.

9. The extendable tubing extension set of claim 8, wherein, in the un-extended configuration, the first and second ends of the tubing cradle are positioned over the top surface of the base.

10. The extendable tubing extension set of claim 8, wherein the tubing cradle is movable, relative to the base, along a first portion of the tubing cradle length.

11. The extendable tubing extension set of claim 10, wherein the tubing cradle is coupled to the base along a second portion of the tubing cradle length.

12. The extendable tubing extension set of claim 8, comprising an adhesive along at least a portion of the bottom surface of the base.

13. An extendable tubing extension set comprising:
a tubing cradle comprising a first end, a second end, and a tubing passage extending through the first and second ends of the tubing cradle, wherein the tubing cradle comprises a tubing cradle length from the first end to the second end of the tubing cradle;
a tubing comprising a proximal end portion, a distal end portion, and a middle portion, wherein the middle portion of the tubing is positioned within the tubing passage with the proximal end portion of the tubing extending beyond the first end of the tubing cradle and the distal end portion of the tubing extending beyond the second end of the tubing cradle;
an un-extended configuration wherein the first end of the tubing cradle is positioned a first distance from the second end of the tubing cradle, and the first distance is less than the tubing cradle length; and
an extended configuration wherein any of the first end or the second end are moved in a direction away from the other of the first end and the second end, such that the first end of the tubing cradle is positioned a second distance from the second end of the tubing cradle, and the second distance is greater than the first distance.

14. The extendable tubing extension set of claim 13, comprising a first fluid connector coupled to a proximal end of the tubing, and a second fluid connector coupled to a distal end of the tubing.

15. The extendable tubing extension set of claim 13, comprising a fluid disconnection device coupled to the tubing.

16. The extendable tubing extension set of claim 15, wherein the fluid disconnection device is coupled between the middle portion of the tubing and the distal end portion of the tubing, between the second end of the tubing cradle and a distal end of the tubing.

17. The extendable tubing extension set of claim 15, wherein the fluid disconnection device is positioned between the second end of the tubing cradle and a distal end of the tubing.

18. The extendable tubing extension set of claim 13, comprising a base having a top surface and a bottom surface, wherein the bottom surface is configured to engage against a patient, and the top surface is configured to engage against the tubing cradle.

19. The extendable tubing extension set of claim 18, wherein the base comprises an arm configured to couple with the tubing.

20. The extendable tubing extension set of claim 18, comprising an auxiliary port coupled to the tubing.
